(19) United States
(12) Patent Application Publication
(10) Pub. No.: US 2009/0300782 A1
(43) Pub. Date: Dec. 3, 2009

(54) Targeted gene addition in stem cells

(75) Inventors: R. Michael Linden, London (GB); Nathalie Dutheil, Astoria, NY (US); Els Henckaerts, New York, NY (US); Gordon Keller, Toronto (CA)

Correspondence Address: CROWELL & MORING LLP;INTELLECTUAL PROPERTY GROUP, P.O. BOX 14300, WASHINGTON, DC, 20044-4300, US

(73) Assignee: MOUNT SINAI SCHOOL OF MEDICINE OF NEW YORK UNIVERSITY, New York, NY (US)

(21) Appl. No.: 11/918,698

(22) PCT Filed: Apr. 18, 2006
(86) PCT No.: PCT/US2006/014391

§ 371 Date: Jul. 13, 2009

Related U.S. Application Data
(60) Provisional application No. 60/672,617, filed on Apr. 18, 2005.

(51) Int. Cl. A01K 67/027; C12N 15/64; C12N 5/06

(52) U.S. Cl. 800/18; 435/455; 435/456; 435/325; 800/13

(57) ABSTRACT

The present invention provides methods for adenoassociated virus-mediated site-specific integration of a transgene into a stem cell. Stem cells having a transgene integrated therein, and differentiated cells generated from the stem cells are also provided.

### CROSS REFERENCE TO RELATED APPLICATION

This application claims the benefit of U.S. application Ser. No. 60/672,617 filed Apr. 18, 2005, the disclosure of which is incorporated herein by reference.

### GOVERNMENT SUPPORT

Portions of this work were supported by grant numbers GM62234-03S1, RO1GM071023 and P20GM075019 awarded by the National Institute of General Medical Sciences of the National Institutes of Health. The United States government may have certain rights in this invention.

### BACKGROUND OF THE INVENTION

Wild type adeno-associated virus (wtAAV) has met the ultimate challenge of maintaining a capacity to propagate its genome without threatening the health of the host organism by adopting the strategy of two alternative pathways during the viral life cycle. First, AAV replicates, killing the host cell, only in the presence of helper factors, which are by themselves deleterious to the host cell. Among those helper functions identified to date are super- or co-infection with viruses like adenovirus and herpes viruses. In the absence of helper functions, wtAAV enters the latent pathway by integrating its DNA site-specifically into the human genome. In this integrated state AAV can stay dormant for many passages with no deleterious effects. The observations regarding the absence of phenotypic changes are based on studies in tissue culture since, as yet, no suitable animal model has been available. To conclude the life cycle, when the host cell is challenged with super-infection by a helper virus, AAV can be rescued from its latent state, initiating replication and highly efficient propagation of the virus, leading to host cell death. This strategy of alternative pathways distinguishes AAV from the autonomous parvoviruses and has led to the establishment of an independent genus within the family of the Parvoviridae, the dependoviruses. Berns (1996) in Fields Virology (eds. Fields et al.) 2173-2197, Lippencourt-Raven, Philadelphia.

wtAAV has a 4.7 kb linear single-stranded genome containing two open reading frames (ORF), flanked by inverted terminal repeats (ITRs). Srivastava et al. (1983) J. Virol. 45:555-564. The right ORF encodes the three capsid proteins, and the left ORF encodes the four non-structural proteins (Rep proteins) that are involved in regulating all aspects of the viral life style. The 145-nt ITRs are the only viral sequences required in cis for DNA replication, packaging of the viral genome into the capsid, and site-specific integration. Within the ITRs, a Rep binding site (RBS) allows for specific recruitment of the large Rep proteins (i.e. Rep 68 and Rep 78) to the origin of replication. Chiorini et al. (1995) J. Virol 69:73334-8. A Rep-specific endonuclease site (terminal resolution site, TRS) is separated from the RBS by a 13 nt-spacer. Brister et al. (1999) J. Virol. 73:9325-36. Together, RBS and TRS can act as a minimal origin for Rep-mediated DNA replication. Smith et al. (1999) J. Virol. 73:2930-7.

It has been shown that site specificity in targeted AAV DNA integration is determined by cellular sequences (Giraud et al. (1994) Proc. Natl. Acad. Sci. USA 91:1003943) and that a 33-nt sequence is necessary and sufficient for this targeted nonhomologous recombination event to occur. Linden et al (1996) Proc. Natl. Acad. Sci. USA 93:7966-72. This 33-nt chromosomal sequence is similar to the minimal viral origin of DNA replication, consisting of an RBS and TRS, suggesting that Rep-mediated DNA replication is involved in the integration mechanism. Complementing this idea was the observation that the viral Rep proteins are required for site-specific integration. Surosky et al. (1997) J. Virol. 7:7951-9.

Biochemical assays have further shown that the Rep proteins can specifically interact with the viral and cellular RBS and TRS motifs to mediate replication and potentially targeted integration of AAV into AAVS1. Kotin (1994) Hum. Gene Ther. 5:793-801. Although all of several isolated viral cellular junctions contain AAVS1 sequences, the immediate transitions from viral to cellular sequences are scattered over a range of approximately 1,000 nucleotides downstream of the TRS-RBS motif within AAVS1. Samulski et al (1991) Embo. J. 10:3941-50. These observations are in agreement with the hypothesis that limited cellular replication is involved in the initial steps of the mechanism underlying AAV site-specific integration.

The target sequence for AAV site-specific integration is closely linked to the muscle-specific genes TNNT1 (encoding slow skeletal muscle troponin T) and TNNI3 (encoding cardiac troponin I). In addition, site-specific AAV DNA integration can result in the formation of TNNT1-AAV junctions. Dutheil et al. (2000) Proc. Natl. Acad. Sci. USA 97:4862-6. It has recently been reported that the AAVS1 RBS is located 17-nt upstream from the translation initiation site of the protein phosphatase 1 regulatory inhibitor subunit 12C gene (PPP1R12C), also called MBS85, that encodes the Myosin Binding Subunit 85 protein. Tan et al. (2001) J. Biol. Chem. 276:21209-1.

As discussed hereinabove, wtAAV has evolved a unique mechanism for integrating its genome site-specifically into human chromosome 19 at AAVS1. In the context of AAV-based strategies for gene delivery, such targeted integration may diminish concerns about mutagenesis due to random integration. However, the question remains whether integration into the AAVS1 site is safe and beneficial. The potential consequences of insertional mutagenesis are of particular concern in fast-dividing embryonic stem (ES) cells.

ES cells are continuously growing stem cell lines of embryonic origin which may be derived from the inner cell mass of developing mammalian blastocysts, and which were initially derived from the mouse blastocyst. Evans et al. (1981) Nature 292:154-6. The distinguishing features of ES cells are the capacity to be maintained and expanded in an undifferentiated state indefinitely in culture while retaining the potential to participate fully in fetal development when reintroduced into the embryo. Bradly et al. (1981) Nature 309:255-6. Maintenance of the pluripotent stem cell phenotype is not cell-autonomous. Embryonic feeder layers or leukemia inhibitory factor (LIF), in the presence of serum, may be used to sustain self-renewal in mouse ES cells. Williams et al. (1988) Nature 336:684-7; Smith et al. (1988) Nature 336:688-90. In serum-free cultures, bone morphogenetic proteins (BMPs) and LIF are needed for ES cell self-renewal. Ying et al. (2003) Cell 115:281-292. Since their establishment in 1981, ES cells have been widely used to create mice with specific genetic deletions, since mutations introduced in mouse ES cells by homologous recombination may be carried into the germ line. Capecchi (1989) Science 244:1288-92. Human ES cells may be maintained in an undifferentiated state by culturing with fibroblast feeder layers in the presence of serum or under serun-free conditions using serum replacement supplemented with basic fibroblast growth factor (bFGF). Culture systems may be based on human feeder layers. Amit et al. (2003) Biol. Reprod. 68:2150-2156. Human ES cells may also be maintained on matrigel or laminin in medium conditioned by mouse embryonic fibroblast feeders (Xu et al. (2001) Nat. Biotechnol. 19:971-974) or in unconditioned medium with bFGF and a BMP antagonist (Xu et al. (2005) Nature Methods 2:185-190.).

ES cells have the unique ability to spontaneously differentiate and to generate a wide range of well-defined cell types under appropriate conditions in culture. Smith (2001) Annu. Rev. Cell Dev. Biol. 17:435-62. The model system for ES cell in vitro differentiation is based on the formation of three-dimensional structures known as embryoid bodies that contain developing cell populations presenting derivatives of all three germ cell layers. Id. Culture conditions have been defined for the in vitro generation of cell types found in the blood, heart, muscle, blood vessels, brain, bone and reproductive system. As a result of this multi-lineage differentiation capacity, ES cells have been widely recognized as a valuable model system for studying the mechanisms underlying lineage specification during the early stages of mammalian development. Odorico et al. (2001) Stem Cells 19:193-204.

The potential of genetic engineering of ES cells has long been recognized. The first reports demonstrated that vectors derived from retroviruses could infect ES cells and that the integrated virus was transmitted through the germ line. Robertson et al. (1986) Nature 323:445-8. However, later analyses revealed that expression from the viral long terminal repeats (LTR) was not active due to transcriptional silencing attributed to trans-acting factors binding to the viral promoters in the LTRs and methylation of the proviral genome and flanking host DNA sequences. A more successful strategy to genetically modify ES cells was found to be homologous recombination between an incoming DNA and its cognate DNA. Wong et al. (1986) Somat. Cell Mol. Genet. 12:63-72. This method has allowed investigators to create knock-out, knock-in, subtle and even conditional 30 mutations in ES cells. Since genome engineering via homologous recombination is quite time-consuming, the search for alternative methods to deliver foreign genes into ES cells has continued. Two recent studies have shown that transgenes delivered to ES cells by lentiviral vectors were not shut off during differentiation and that the transgene was expressed in multiple tissues of chimeric animals generated by transfer of lentiviral vector-transduced ES cells in blastocysts. However, in both studies transgene expression was related to the number of proviral copies; in some clones up to twelve copies were observed. Hamaguchi et al. (2000) J. Virol. 74:10778-84.

Data in the literature on the infectivity of ES cells with AAV is discouraging, showing at best minimal infection efficiency with one serotype, and a lack of stable transgene expression (Smith-Arica et al. (2003) Cloning Stem Cells 5:51-62) or random transgene integration (Wei et al. (2004) Preclinica 2:262-266). Random integration, particularly of multiple copies, is a concern in the development of ES cell-based cell replacement therapies. Random integration by retrovirally delivered transgenes implies that the chromosomal context and thus expression of a transgene will vary between vector-transduced cells. Many of these studies have indeed been hampered by shutdown of transgene expression as soon as differentiation is initiated. A second consideration concerning random integration by retrovirally delivered transgenes is the risk of insertional mutagenesis. While in differentiated cells the potential risk associated with insertional mutagenesis is apparently negligible, in ES cells, which could be expanded, differentiated and ultimately used as a source for transplantation, this aspect has not heretofore been addressed. The autogenesis potential of rapidly dividing stem cells has now been tragically documented in humans by the emergence of leukemia as a result of retrovirally mediated gene therapy of X-linked SCID in an otherwise highly successful clinical trial. Therefore, a need exists to develop an efficient and safe method to genetically modify stem cells.

### SUMMARY OF THE INVENTION

The present invention provides a method for site-specific integration of a transgene into the genome of an embryonic stem (ES) cell comprising introducing into the ES cell an adeno-associated virus (AAV) vector containing the transgene and a Rep protein or a nucleic acid encoding a Rep protein.

The present invention further provides a method for site-specific integration of a transgene into the genome of an adult stem cell comprising contacting the adult stem cell with an AAV vector comprising the transgene and a Rep protein or a nucleic acid encoding a Rep protein.

In another embodiment, the present invention provides a stem cell having a transgene integrated into the genome of the stem cell by the method of the present invention. Differentiated cells and tissues generated from such stem cells are also provided.

An animal modified to have a stem cell produced by the method of the invention introduced therein, or a differentiated cell or tissue derived from said stem cell introduced therein is also provided.

In another embodiment, the present invention provides an in vivo assay system comprising a non-human animal having introduced therein a cell modified by the method of the present invention.

The present invention further provides a transgenic non-human animal and progeny thereof wherein said transgenic animal comprises a transgene integrated into AASV1.

### BRIEF DESCRIPTION OF THE DRAWINGS

FIG. 1 is a schematic showing the IRS/RBS motifs present in human and mouse AAVS1 in the context of Mbs85 and neighboring genes.

### DETAILED DESCRIPTION OF THE INVENTION

The present invention provides a method for site-specific integration of a transgene into the genome of a mammalian ES cell comprising introducing into the ES cell an AAV vector containing the transgene and a Rep protein or a nucleic acid encoding a Rep protein. In particular, the present invention provides an efficient method for the site-specific integration of a transgene into the genome of an ES cell.

In a preferred embodiment, the ES cell is a human or a mouse ES cell. ES cells may be obtained commercially or isolated from blastocysts by methods known in the art, as described for example by U.S. Pat. No. 5,843,780; Thompson et al. (1998) Science 282:1145-1147; U.S. Pat. No. 6,492,575; Evans et al. (1981) Nature 292:154-156; and Reubinoffet al. (2000) Nature Biotech. 18:399.

The method described herein may also be used to deliver a transgene to an adult, i.e. somatic, stem cell. Adult stem cells include, for example, hematopoietic stem cells, bone marrow stromal stem cells, adipose derived adult stem cells, olfactory adult stem cells, neuronal stem cells, skin stem cells, and so on. Adult stem cells have a similar ability as ES cells to give rise to many different cell types, but have the advantage that they can be harvested from an adult.

The AAV vector containing the transgene comprises a pair of AAV inverted terminal repeats (ITRs) which flank at least one cassette comprising a transgene under the control of a promoter. Transgene in this context refers to any nucleotide sequence which is not native to AAV. The AAV ITRs, in combination with a Rep protein, confer infectivity and site-specific integration without toxicity. The ITRs may be derived from any AAV serotype, including AAV1-9. A preferred embodiment utilizes serotype 2. The AAV ITRs and methods of obtaining the ITRs are well-known in the art and disclosed, for example, in U.S. Pat. No. 5,252,479. The vectors may further contain sequence elements which facilitate expression and cloning, for example enhancers and selectable markers. Recombinant AAV vectors for noncytotoxic gene transfer and methods for making such vectors are known in the art and disclosed for example in U.S. Pat. Nos. 6,632,670; 5,252,479; 5,173,414 and Kotin et al. (1994) Human Gene Therapy 5:793-801. Methods for producing stocks of recombinant AAV are known in the art and disclosed for example by Zolotukhin et al. (2002) Methods 28:158-167; Zolotukhin et al. (1999) Gene Ther. 6:973-985; and Grimm et al. (1998) Hum. Gene Ther. 9:2745-60 and reviewed by Zolotukhin (2005) Hum. Gene Ther. 16:551-557. Viral vector systems having hybrid serotypes and custom AAV capsids are also included in the present invention and disclosed for example by Choi et al. (2005) Curr. Gene Ther. 5:299-310; Gas et al. (2005) Curr. Gene Ther. 5:285-297; Muzyczka et al. (2005) Hum. Gene Ther. 16:408-416; and Buning et al. (2004) Cells Tissues Organs 177:139-150. The AAV vector may comprise an AAV capsid comprising capsid proteins from any of the AAV serotypes, or combinations thereof. Pseudotyped vectors comprising the AAV IRs from one serotype and capsid proteins from a different serotype are included herein.

The transgene is a nucleic acid sequence that is heterologous to AAV. For example, the transgene may encode a marker or reporter molecule, protein, peptide, antisense nucleic acid, or catalytic RNA. The transgene may encode a naturally or non-naturally occurring molecule, including for example a chimeric or hybrid polypeptide. In a preferred embodiment, the transgene encodes a product that is useful for the treatment of a disease or disorder.

A Rep protein or nucleic acid encoding a Rep protein used in the present method mediates the site-specific integration of the transgene. The Rep protein may be any AAV Rep protein or combination of AAV Rep proteins or a Rep protein variant or fragment that is sufficient to mediate site-specific integration. The term Rep protein as used herein also includes Rep-like proteins such as the human herpes virus 6 (HHV-6) Rep (Thompson et al. (1994) Virology 204:304-311) and goose parvovirus (GPV) Rep 1 (Smith et al. (1999) J. Virol. 72:2930-2937) and fragments thereof that are sufficient to mediate site-specific integration. Hybrids of Rep proteins or fragments thereof with Rep-like proteins or fragments thereof are also included and disclosed for example by Yoon et al. (2001) J. Virol. 75:3230-3239. The Rep protein may be derived from any AAV serotype, and includes native, variant and chimeric forms of a Rep protein. Variants that maintain the function of mediating integration are well-known in the art (see, e.g. Yoon et al. (2001) J. Virol. 75:3230-3239) or can be ascertained by mutational analyses. In a preferred embodiment the Rep protein is Rep 68 or Rep 78 or a fragment thereof that is sufficient to mediate site-specific integration. In a preferred embodiment the Rep protein comprises the amino-terminal 208 amino acids of Rep 78.

In accordance with the present method, a Rep protein or a nucleic acid encoding a Rep protein is provided to the ES cell. A nucleic acid encoding a Rep protein may be provided in trais by co-transfection of the AAV vector with a Rep-expressing construct, which may be in the form of a plasmid, phage, transposon, cosmid, virus or virion. Such constructs are known in the art and disclosed for example in U.S. Pat. Nos. 6,632,670; 5,952,221; 5,139,941; Samulski et al. (1989) J. Virol. 63:3822-3828 and McCarty et al. (1991) J. Virol. 65:2936-2945. The ES cell may be stably transformed by a nucleic acid encoding a Rep protein prior to introduction of an AAV vector. A nucleic acid encoding a Rep protein may also be provided in cis by methods known in the art, for example by a vector that directs the delayed expression of the rep sequences as disclosed in U.S. Pat. No. 6,294,370 or a vector in which a rep coding region is sited outside the ITRs, as disclosed by Linden et al. (1997) Gene Therapy 4:4-5.

A Rep protein may be provided to an ES cell by methods known to those of ordinary skill in the art including methods using encapsulating media such as cationic lipid reagents, or methods of calcium phosphate precipitation, electroporation and microinjection. Additional methods that may be used include protein transduction methods in which the Rep proteins are conjugated to peptides known as protein transduction domain (PTPs) or cell penetrating peptides (CPPs). Such peptides include, for example, the herpes simplex virus (HSV) type 1 protein VP22, the human immunodeficiency virus (HIV-1) transactivator TAT protein, polyarginine and polylysine. Methods of protein transduction are known in the art and are reviewed by Noguchi et al. (2006) Acta Med. Okavama 60:1 -11, and Wadia et al. (2002) Curr. Opin. Biotechnol. 13:52-56. The peptides may be covalently cross-linked to the Rep proteins or synthesized as fusions with the Rep proteins. Other methods for delivering the Rep proteins into ES cells include a non-covalent peptide-based method using an amphipathic peptide as disclosed for example by Morris et al. (2001) Nat. Biotechnol. 19:1173-1176 and U.S. Pat. No. 6,841,535 and indirect polyethylenimine cationization as disclosed for example by Kitazoe et al. (2005) J. Biochem. (Tokyo) 137:643-701.

It has been discovered in accordance with the present invention that transduction efficiency of stem cells is enhanced by the use of double-stranded AAV vectors (dsAAV) in the present method. Such dsAAV is known in the art and disclosed by Wang et al. (2003) Gene Ther. 10:2105-2111, and has a deletion of the terminal resolution site (TRS) in one ITR. As a result, this ITR cannot be resolved during replication, leading to the generation of replication intermediates that are 2× in length with two complementary single strands that are separated by the partially deleted ITR. Accordingly, in one embodiment of the present invention the AAV vector comprises a pair of ITRs flanking a cassette comprising a transgene under the control of a promoter, in which one of the ITRs has a deletion of the TRS.

In accordance with the present invention, the method is preferably performed at multiplicities of infection of 10³-10⁶ genomes per cell. The undifferentiated ES cells are preferably maintained under conditions that allow maintenance of healthy colonies in an undifferentiated state. For example. human ES cells may be maintained on a feeder layer such as irradiated mouse embryonic fibroblasts in the presence of serum, or with serum replacement in the presence of bFGF, or in medium conditioned by mouse embryonic fibroblasts, or under serum free conditions using human feeder layers derived from, for example, human embryonic fibroblasts, fallopian tube epithelial cells or foreskin.

Mouse ES cells may be maintained, for example, on a feeder layer such as irradiated mouse embryonic fibroblasts in the presence of serum and LIF, or on gelatin plates without feeder cells in the presence of LIF and serum.

In another preferred embodiment, ES cells are maintained on a solubilized basement membrane preparation such as Matrigel™ (Kleinman et al (1982) Biochem. 21:6188; Becton Dickinson Biosciences). Methods for maintaining ES cells are known in the art and disclosed for example by Williams et al. (1988) Nature 336:684-7; Smith et al. (1988) Nature 336:688-90; Ying et al. (2003) Cell 115:281-92; Amit et al. (2003) Biol. Reprod. 68:2150-2156; and Amit et al. (2000) Developmental Biology 227:271-278.

The method of the present invention results in site-specific integration of the transgene at the AAVS1 locus of the ES cell genome (human chromosome 19 at 19 q 13.4; mouse chromosome 7; 9.0 cM). The ES cells having the integrated transgene undergo normal embryoid body (EB) development and retain the capacity to differentiate into multiple cell types. Expression of the transgene is maintained throughout differentiation. Further, the ES cells having the integrated transgene maintain the capacity to generate cells of multiple lineages.

Stem cells having a transgene integrated therein as made by the method of the present invention are useful, inter alia, for generating transgenic non-human animals, for generating differentiated cells and tissues having a transgene integrated therein, for studying differentiation of stem cells, for evaluating strategies for safe and effective gene targeting in stem cells, and for targeted therapeutic gene transfer.

Methods for generating differentiated cells from stem cells are known in the art. The model system for ES cell in vitro differentiation is based on the formation of three dimensional structures known as embryoid bodies (EBs) that contain developing cell populations presenting derivatives of all three germ layers and is disclosed in the art, for example by Keller (1995) Curr. Opin. Cell Biol. 7:862-869.

For example in one embodiment prior to differentiation, ES cells are removed from feeder cells prior to differentiation by subcloning the ES cells directly onto a gelatinized culture vessel. Twenty-four to 48 hours prior to the initiation of EB generation, ES cells are passaged into IMDM-ES. Following 1-2 days culture in this medium, cells are harvested and transferred into liquid medium (IMDM, 15% FBS, glutamine, transferrin, ascorbic acid, monothioglycerol and protein free hybridoma medium II) in Petri-grade dishes. Under these conditions, ES cells are unable to adhere to the surface of the culture dish, and will generate EBs.

Culture conditions are known in the art for the differentiation to cell types found in blood (Wiles et al. (1991) Development 111:259-67), heart (Maltsev et al. (1993) Mech. Dev. 44:41-50), muscle (Rohwedel et al. (1994) Dev. Biol. 164:87-101), blood vessels (Yamashita et al. (2000) Nature 408:92-96), brain (Bain et al. (1995) Dev. Biol. 168:342), bone (Buttery et al. (2001) Tissue Eng. 7:89-99) and reproductive system (Toyooka et al. (2003) Proc. Natl. Acad. Sci. USA 100:11457-11462).

The differentiated cells and/or tissue generated therefrom may be introduced in an animal for therapeutic purposes. Accordingly, in another embodiment the present invention provides an animal comprising differentiated cells having a transgene integrated into the AAVS1 locus thereof, or comprising a tissue generated from such cells. In a preferred embodiment the differentiated cell is a hemotopoietic cell, endothelial cell, cardiomyocyte, skeletal muscle cell or neuronal cell. The cells or tissues may be transplanted into the animal by methods known in the art.

The present invention also provides a transgenic non-human animal and progeny thereof wherein said transgenic animal comprises a transgene integrated into AAVS1. In a preferred embodiment, the animal is a mouse. Such transgenic animals provide an in vivo system for studying the consequences of disruption of the AAVS1-associated gene cluster, and for assessing the safety, efficacy and regulatability of AAV-mediated delivery of transgenes. Transgenic mice having a marker gene such as the gene encoding GFP are particularly useful for testing site-specific integration of a transgene, since successful integration results in loss of the marker due to disruption of the marker gene.

Methods for producing transgenic mice are well-known in the art. For example, the transgenic mouse may be obtained by injecting ES cells having a transgene integrated therein into blastocysts, which are then implanted into pseudopregnant females and allowed to develop to term. Recipient mouse strains having a different fur color then the strain from which the ES cell is derived may be used to facilitate the identification of chimeric mice. The inclusion of a marker gene as a transgene facilitates the identification of donor ES cell derived cells in tissues other than the fur, e.g., blood.

All references cited herein are incorporated herein in their entirety.

The following examples serve to further illustrate the present invention.

### EXAMPLE 1

### Characterization of Mouse AAVS1 Ortholog

The nonpathogenic human adeno-associated virus (AAV) has developed a mechanism to integrate its genome into human chromosome 19 at 19q13.4 (termed AAVS1), thereby establishing latency. U.S. Pat. No. 5,580,703; Dutheil et al. (2000) Proc. Natl. Acad. Sci. USA 97:4862-66. This example demonstrates that the chromosomal signals required for site-specific integration are conserved in the mouse genome proximal to the recently identified Mbs85 gene. These sequence motifs can be specifically nicked by the viral Rep protein required for the initiation of site-specific AAV DNA integration. Furthermore, these signals can serve as a minimal origin for Rep-dependent DNA replication. In addition, the mouse Mbs85 proximal promoter was isolated and transcriptional activity was shown in three mouse cell lines.

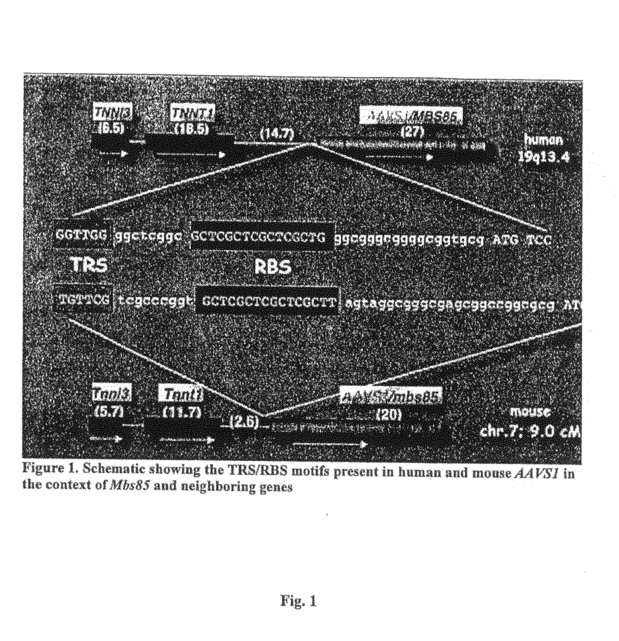

By using MBS85 (myosin binding subunit 85) exon sequences, the National Center for Biotechnology Information mouse database was analyzed for similarities to the human AAVS1 locus as described by Altschul et al. (1997) Nucleic Acids Res. 25:3389-3402. This analysis revealed a homology of 90% between the 5′ end of the human MBS85 cDNA and the 969-nt mouse cDNA clone AK010836, which contains a sequence homologous to the human TRS-RBS motifs as well as the Mbs85 initiation codon (separated by 25 nt). FIG. 1. A simian AAVS1 locus containing the corresponding upstream region and a TRS-RBS motif has recently been isolated from the African green monkey genome by Amiss et al. (2001) Methods Mol. Biol. 175:455-469. AAVS1 is located 14.9 and 36 kb centromeric to the slow skeletal troponin T (TNNT1) and cardiac troponin I (TNNI3) genes, respectively. Dutheil et al. (2000) Proc. Natl. Acad. Sci. USA 97:4862-4866. The mouse Tnni3 and Tnnt1 genes are located on chromosome 7, in a region previously shown to be syntenic to the human chromosome 19 region that contains AAVS1. Blake et al. (2000) Nucleic Acids Res. 28:108-111. The Celera discovery system was used to search the Celera mouse genome assembly with the mouse Tnni3 and Tnnt1 genes, the AK010836 cDNA, and the human MBS85 genomic sequence. All of these sequences specifically matched the same scaffold (500 kb) in the Celera database. The mouse Mbs85 is located on chromosome 7 and is separated by only 2.5 and 16 kb from the Tnnt1 and Tnni3 genes, respectively. The Celera map revealed a gene 3.1 kb downstream of MBS85, designated DRC3, the mouse homolog of which is located 2.1 kb downstream of the Mbs85 gene.

Three mouse expressed sequence tag clones (AA021750, AW911639, and BE847281) containing Mbs85 were sequenced and assembled. The resulting 3.1-kb mouse cDNA was 77% identical to the human MBS85 cDNA. The mouse Mbs85 gene spans 20 kb of genomic sequences, and the 2.3-kb predicted open reading frame is composed of 22 coding exons. Thus, the mouse and the human homologs of MBS85 display the same overall genomic organization. The deduced mouse Mbs85 protein sequences is 781 amino acids in length and is 86% identical to its human counterpart. Tan et al. (2001) J. Biol. Chem. 276:21209-21216.

To access the distribution of Mbs85 mRNAs, a mouse poly(A) multiple tissue Northern blot (Clontech, Palo Alto, Calif.) was hybridized to a mouse Mbs85 cDNA probe consisting of exons 5 to 22. As is observed in a human multiple tissue Northern blot (Tan et al. (2001) J. Biol. Chem. 276:21209-21216), a single mRNA of approximately 3.1 kb is highly expressed in heart and testis, and to a lesser extent in kidney, brain, liver, and lung.

To determine if Rep68 can specifically nick the putative mouse TRS, double-stranded and partially single-stranded 5′ end-labeled origin substrates were incubated with purified His-tagged Rep68 proteins in a cell-free endonuclease assay as described by Yoon et al. (2001) J. Virol. 75:3230-3239. Rep68 nicked the AAV, human, and mouse TRS substrates releasing an expected 14-nt labeled fragment. Nicking is Rep68 dependent since no cleavage of the AAV, human, or mouse origin substrates is observed when an endonuclease-negative mutant is used (Rep⁶⁸Y156F). Smith et al. (2000) J. Virol. 74:3122-3129; Yoon et al. (2001) J. Virol. 75:3230-3239. Substitution of the two thymidine residues within the mouse TRS sequence resulted in an expected loss of specific Rep-mediated cleavage.

Origin interactions by Rep are thought to represent the initiating steps of integration. Ward et al. (2001) J. Virol. 75:10250-10258. To test whether the mouse TRS-RBS sequence could also serve a similar function, cell-free DNA replication assays were performed as described by Ward et al. (1994) J. Virol. 68:6029-6037. Linearized substrates containing the AAV, human, or putative mouse origin in a pBluescript backbone were incubated with HeLa cell extracts in the presence or the absence of purified His-tagged Rep68 (75 ng) and [α³²P]dCTP. Rep68 initiated replication on templates containing the AAV, human, or mouse origin but not on the vector DNA alone. In all cases, replication was Rep dependent. The human and mouse 5′ untranslated regions were further compared. It has been reported that the human AAVS1 fragment located 74 to 426 upstream of the translation initiation codon is sufficient to drive the expression of a reporter gene following transient transfections in both 293 and HeLa cells. Lamartina et al. (2000) J. Virol. 74:7671-7677.

Alignment of the human and mouse sequences upstream of the ATG revealed an overall 62% identity in the putative promoter region. Several conserved putative cis-acting DNA elements (i.e., Sp1, CRE/ATF) indicate the presence of a TATA-less promoter and common regulatory mechanisms for the expression of the human and mouse MBS85 genes.

Mouse cell lines expressing Mbs85 were identified. Total RNAs were extracted from C2C12, NIH 3T3, and N2A cell lines (Tel-Test, Friendswood, Tex.) Northern blots hybridized to the mouse Mbs85 ex5-22 cDNA probe revealed a unique 3.1-kb transcript in all three cell lines.

To test the 324-bp NaeI fragment containing the RBS and TRS motifs for transcriptional activity, it was cloned into the pDsRed2.1 promoterless red fluorescent protein vector (Clon-tech) in both the sense and antisense orientation. C2C12, N2A, and NIH 3T3 cells were transfected and fixed 45 hours posttransfection with 3.7% paraformaldehyde, and the slides were mounted in vectashield mounting medium with DAPI (4′,6′-diamidino-2-phenylindole) (Vector Laboratories, Burlingame, Calif.). The sense, but not the antisense, construct shows transcriptional activity in all three cell lines. These results were confirmed by fluorescence-activated cell sorter analysis.

This example demonstrates that the target for AAV site-specific integration is not restricted to primates but is also present in the mouse genome in a region that is syntenic to the human chromosome 19 region containing AAVS1.

Currently, Rep interactions with a minimal origin are defined by specific binding to the RBS followed by site- and strand-specific nicking at the TRS. This example demonstrates that the IRS and RBS motifs present in the 5′ untranslated region of the mouse Mbs85 gene can act as a substrate for Rep-mediated nicking and as a functional Rep-dependent origin.

It also demonstrates that a region containing the TRS-RBS motif upstream of the mouse Mbs85 ATG contains regulatory elements sufficient to drive the expression of a reporter gene in vitro.

The following materials and methods were used in the foregoing example.

To determine tissue distribution of Mbs85 mRNAs in the mouse, a multiple tissue Northern blot derived from mouse tissues was hybridized with a cDNA probe consisting of exons 5 to 22 (ex 5-22) of the mouse Mbs85 cDNA. The ex 5-22 probe was generated by digestion of clone BF540586 with EcoRI/HindIII.

For cell-free endonuclease assays, fully double-stranded and partially single-stranded origin substrates (as a specificity control) (5 fmol) containing the AAV, human (his), mouse (mS1), and mouse origin mutant (mS1mut) sequences were incubated for 45 min at 37° C. in either the absence or presence of 1 pmol of AAV Rep68 protein or 1 pmol of AAV Rep68 endonuclease mutant (Y156F). 5′ end-labeled marker oligonucleotides corresponding in sequence and length to the expected reaction product (14 nt) were used. Synthetic oligonucleotide substrates were used in the nicking and replication assays. The TRS-containing strand was first kinase labeled and then annealed to its complementary strand.

In the cell-free DNA replication assay, the AAV, human (hS1) and mouse (mS1) origins (consisting of the TRS and RBS sequences) were cloned into pBluescript via XbaI and SaII sites. Prior to the assay, plasmids were linearized with XmnI. Each linear, origin containing substrate was incubated in the presence or absence of AAV Rep68 protein.

Expression of Mbs85 was determined by Northern blot analyses. The Northern blot of C2C12, N2A, and NIH 3T3 cells was hybridized with a cDNA probe consisting of exons 5 to 22. The blot was stripped and rehybridized with a β-actin cDNA probe. Transcriptional activity of the mouse Mbs85 proximal promoter was determined as follows. Plasmid pDsRed2-N1 (red fluorescent protein under the cytomegalovirus promoter; Clontech) and the sense and antisense plasmids were transfected into C2C12, NIH 3T3, and N2A cells. Forty-five hours posttransfection, the cells were visualized for redfluorescent protein expression and DAPI staining by using an epifluorescent microscope (Leica DMRA2) and a Hamamatsu digital camera.

The foregoing results are published as Dutheil et al. (2004) J. Virol. 78:8917-8921, the disclosure of which is incorporated herein in its entirety.

### EXAMPLE 2

### Materials and Methods

The following materials and methods were used in subsequent examples.

Plasmid constructs. The conventional rAAV-GFP vector plasmid (pTRUF11) is described by Zolotuldin et al. (1996) J. Virol. 70:46464654 and Zolotukhin et al. (1994) Gene Ther. 6:973-985. It carries the humanized green fluorescent protein (hGFP) sequence under the control of the hybrid CMVie enhancer/chicken β-actin promoter (CBA) flanked by the ITRs of AAV2. Plasmid pAV2, used to produce wild type AAV2 virus, is described by Laughlin et al. (1983) Gene 23:65-73. Plasmids pXYZ1, pDG, pXYZ5, pDG-AAV8, pDG-AAV9 were used as helper to produce AAV serotypes 1, 2, 5, 8 and 9 respectively. These plasmids were all derived from pDG (Grimm et al. (1998) Human Gene Ther. 10:2745-2760) and carry the genes required for rAAV packaging. pXYZ1 and pXYZ5 are described by Zolotukhin et al. (2002) Methods 28:158-167. pDG-AAV8 and pDG-AAV9 were constructed using the AAV8 capsid sequence isolated from non-human primates in the laboratory of K. R. Clark. The AAV9 capsid sequence is described by Gao et al. (2004) J. Virol. 78:6381-6388.

### Wt and Recombinant Adeno-Associated Virus Production.

The rAAV production and purification schemes were based on the protocol described by Zolothukin et al. (1999) supra. Briefly, 293-T cells (ATCC, Manassas, Va.) were cotransfected with pTRUF11 together with the helper plasmid. After 72 hours, the virus was purified from cell crude lysates over a density gradient made of iodixanol (Optiprep, Greiner Bio-One Inc., Longwood, Fla.). Serotype 2 virus stocks were additionally purified by affinity chromatography using heparin-agarose type I (Sigma-Aldrich Inc., St-Louis, Mo.) as a matrix. Virus samples were next concentrated and formulated into lactated Ringer's solution (Baxter Healthcare Corporation, Deerfield, Ill.) using a Vivaspin 20 Centrifugal concentrators 50K MWCO (Vivascience Inc., Carlsbad, Calif.).

Wild-type AAV2 was produced following the same protocol, using pAV2 instead of pTRUF11.

Maintenance and infection of ES cells. Mouse ES cells (CCE and E14) were maintained in 6-well plates on irradiated mouse embryonic feeder cells in DMEM medium (DMEM-ES) containing 1% L-Glutamine, 2.5% Hepes buffer, 15% fetal bovine serum (FBS, pretested for maintenance of ES cells), 1% Leukemia Inhibitory Factor (LIP—medium conditioned by CHO-LIF cells), and monothioglycerol (1.5×10⁻⁴M). Cultures were monitored daily and cells were passaged every 2-3 days. For passaging, ES cells were trypsinized (0.25% trypsin, 0.1% EDTA), washed and approximately 10% of the cells were replated on fresh feeder cells. Cells were maintained in 37° C. incubators at 5% CO₂.

For feeder depletion prior to infection, ES cells were cultured for 1 passage in wells of 6-well plates coated with a 0.1% solution of gelatin and containing DMEM-ES medium.

Cells were harvested from this culture vessel, counted and seeded in gelatin-coated, DMEM-ES-containing 96-well plates at a density of approximately 10,000 cells per well.

Twenty four hours later, cells from a couple of representative wells were counted in order to calculate the amount of virus needed to infect every well at a multiplicity of infection of 10⁶. ES cells were then infected with single or double strand recombinant AAV2-GFP viruses, resuspended in 30 □1 of DMEM-ES medium. Infections were performed at 37° C.; plates were shaken by hand every 15 minutes. After 1 hour, 70 □1 of fresh medium was added and plates were placed back in the incubator. ES cells were incubated for 48 hours without removing the virus-containing medium.

Generation of EBs from ES cells. The capacity of ES cells to differentiate into multiple cell lineages can be reproduced in culture where ES cells can produce a wide range of well-defined cell types. The model system for ES cell in vitro differentiation is based on the formation of three-dimensional structures known as embryoid bodies that contain developing cell populations presenting derivatives of all three germ cell layers. Keller et al. (1995) Curr. Opin. Cell Biol. 7: 862-869.

Prior to differentiation, ES cells were removed from the feeder cells by subcloning the ES cells directly onto a gelatinized culture vessel. Twenty-four to 48 hours prior to the initiation of EB generation, ES cells were passaged into IMDM-ES. Following 1-2 days culture in this medium, cells were harvested and transferred into liquid medium (IMDM, 15% FBS, glutamine, transferrin, ascorbic acid, monothioglycerol and protein free hybridoma medium II) in Petri-grade dishes. Under these conditions, ES cells are unable to adhere to the surface of the culture dish, and will generate EBs. Keller et al. in Hematopietic Stem Cell Protocols (eds. Klug et al.) 209-230, Humana Press, Inc., Totowa.

Generation of hematopoietic cells, endothelial cells, cardiomyocytes, skeletal muscle and neuronal cells from EBs. Developing hematopoietic precursors within EBs can be identified and studied in a standard colony-forming cell (CFC) assay. After harvest and dissociation (trypsin or collagenase treatment, depending on the duration of EB development), cells were mixed into the methylcellulose-containing medium with specific hematopoietic cytolines, and aliquots were plated in 35×10 mm Petri-grade dishes, which were incubated at 37° C. for various periods of time. Colonies that developed from the hematopoietic precursors were scored between 5-10 days following the initiation of culture. The types of precursors present depend on the age of the EBs. The changing precursor populations provide the basis for defining the three different stages of EB hematopoietic development The earliest stage, the hemangioblast stage, contains the blast-CFC able to generate both endothelial and hematopoietic progeny. EBs at the next stage contain primitive erythroid (E^{P}), definitive erythroid (E^{d}), macrophage, bipotential E^{d}/Mac, bipotential E^{d}/megakaryocyte (Mega), and multipotential precursors. The multilineage definitive stage EBs contain E^{d}, bipotential Ed/mast cell (Mast), Mast, bipotential E^{d}/Mega, Mega, bipotential E^{d}/Mac, Mac, neutrophil (Neut), bipotential Mac/Neut, and multipotential precursors.

To assess the vascular potential of the developing EBs, Flk-1+ cells isolated from day 3-EB differentiation cultures were cultured in collagen gels and analyzed 10 days later. The cells formed vascular sprouts that expressed PECAM-1 (CD31).

Cardiomyocyte potential was analyzed by moving EBs from serum-containing to serum-free medium. Cultures were monitored over a 2- to 7-day period for the development of beating masses. To confirm that the cells were of the cardiomyocyte lineage, aggregates were analyzed for expression of the cardiac specific form of Troponin T. Cells within the masses expressed this marker.

EBs generated in the absence of serum were cultured on gelatin coated six-well-plates and monitored for neurite outgrowth, indicative of neurectoderm differentiation. EBs with visible neurites were transferred to glass cover-slips and stained for B-III tubulin expression. The neurites expressed abundant levels of B-III tubulin demonstrating their neuronal nature. Using conditions described by Rohwedel et al., supra, it was shown that cells with skeletal muscle morphology also develop in these cultures.

Establishment of cloning techniques in mouse ES cells. Since clonality is a prerequisite to analyze AAV-mediated integration events, a cloning technique was developed that would allow for the isolation of clean single-cell derived ES cell clones. A cost-effective way to do this was to generate GFP-expressing ES cells based on transfection. ES cells were transfected, grown on neo^{r} MEF at 50-70% confluency, with pTRUF1, a plasmid that contains the “humanized” GFP (hGFP) gene (Zolotukhin et al. (1996) J. Virol. 4646-4654) and a neomycin resistance cassette flanked by the AAV terminal repeats. Forty-eight hours after transfection, cells were trypsinized and analyzed with flow cytometry (FACS) for transfection efficiencies (±90% of the cells were GFP-positive when transfections were executed with Lipofectamin 2000). Part of the cells were seeded onto fresh neo^{r} feeders and G418 selection was started. Since ES cells could not be single cell sorted, ES colonies were aspirated. These colonies originate from a single cell and can thus be considered clonal. For short periods of G418 selection (e.g. three days), resistant ES colonies were well-spread and could easily be aspirated. For longer selection periods (e.g. two weeks) in which selective colonies were expanded and passaged, single ES colonies were aspirated, trypsinized and seeded in one well of a 24-well plate. The newly developing colonies were now well-spread and single clones could easily be aspirated. GFP-positive colonies, established with this cloning technique, showed homogeneous GFP-expression profiles when analyzed with FACS.

### Infectivity of Mouse ES Cells.

Generation of rAAV. Recombinant viruses of the AAV serotypes 1, 2, 4, 5, 8 and 9 were generated using transfection methods in either triple flasks or ten-layered cell factories. Recombinant AAV contains the marker genes neomycin and GFP flanked by the AAV-ITRS. The serotypes 1, 4, 5, 8 and 9 were generated using the “pseudotyping” approach in which the recombinant genome is flanked by the AAV2 ITRs and the different serotype capsids are packaged by the AAV2 REP. Typically, approximately 2×10¹³ genome containing particles (gcp) per triple flask were produced.

A “double-strand” or dsAAV (in this case containing CMV-EGFP) was produced which is different from traditional viruses in that it has a deletion of the TRS in one ITR. As a result, this ITR cannot be resolved during replication, which leads to the generation of replication intermediates that are 2× in length with two complementary single strands that are separated by the partially deleted ITR. If the total length of these intermediates does not exceed the full length of wtAAV they can be packaged similarly to traditional recombinant viruses. However, when this DNA enters the nucleus it is hypothesized that the complementary strands can anneal, resulting in DNA structures that can directly be transcribed. It is thought that this strategy circumvents the rate-limiting step of second-strand synthesis of traditional recombinant AAV DNA that is believed to underlie the delayed onset of transduction by AAV. Impressively, when these viruses are used for in vivo transduction assays, the expression of the transgene was accelerated and transduction was significantly enhanced (e.g. in contrast to 5% of hepatocytes transduced with traditional viruses, dsAAV infection led to 80-90% of transduced hepatocytes).

### EXAMPLE 3

### Infection of Mouse ES Cells with AAV

In this example, infection experiments were performed using recombinant AAV1, 2 and 5 GFP viruses to infect CCE and E14 cells. Infections at different MOIs were performed on small ES colonies, cultured on gelatin. Flow cytometric analysis of GFP was used to determine transduction efficiencies. Transduction efficiency was measured as the number of GFP-expressing cells present in the cultures 48 hours post-infection. Infections with rAAV2 at a multiplicity of infection (MOI-gcp/cell) of 10⁶ resulted in GFP-expressing ES cells. Infection of ES cells by the other serotypes was not detectable. Experiments were expanded with rAAV2 and transduction efficiencies of single strand (ss) versus ds virus were compared. As shown in Table 1, infections with ss AAV2 consistently yielded about 1% GFP-positive cells and infections with ds AAV2 significantly increased the number of transduced cells. These data indicate that a sufficient number of cells are infected but that the onset of transduction is delayed when ss virus is used. Thus, this example shows that mouse ES cells can be infected with AAV2. C-kit expression and alkaline phosphatase staining were similar in control and infected cell populations, indicating that ES cells could withstand these relatively high multiplicities of infection. These markers indicate that the cultures consisted of undifferentiated, self-renewing ES cells. Similar results were obtained for E14 cells.

TABLE 1

| Mouse | ssAAV2-GFP | dsAAV2-GFP |
|---|---|---|
| CCE | 0.87 ± 0.23 (n = 7) | 12.92 ± 1.16 (n = 6) |
| E14 | 0.97 ± 0.13 (n = 4) | 7.66 ± 0.92 (n = 4) |

Transduction of embryonic stem cells is indicated in % GFP-positive cells. Cells were infected at an M.O.I. of 10⁶. The transduction efficiency was determined by FACS analysis performed 48 hours post-infection.

Subsequently, these experiments were expanded to include additional serotypes, AAV8 and AAV9. As with the previous serotypes, AAV8 and AAV9 were “pseudotyped”, i.e. these vectors contain the AAV2 ITRs and the identical transgene as used earlier. These genomes were packaged into the AAV8 and AAV9 capsids, respectively. In these experiments both single-stranded (ss) as well as double-stranded (ds) vectors of serotypes, AAV1, AAV2, AAV5, AAV8 and AAV9 were used.

Infections at an MOI of 10⁶ were performed on small mouse ES colonies (CCE), cultured on gelatin. Transduction efficiency was determined as the number of GFP expressing cells present in the cultures 48 hours post-infection. As can be seen in Table 2, with the exception of AAV5 (ss and ds), infections of mouse ES cells with ds vectors of all serotypes resulted in significant transduction.

TABLE 2
Transduction efficiencies: percentage of GFP-expressing
cells as determined by flow cytometry

| | Single-stranded | Double-stranded |
|---|---|---|
| AAV1 | 0.11% | 46.37% |
| AAV2 | 7.11% | 16.58% |
| AAV5 | 0.20% | 0.23% |
| AAV8 | 0.59% | 10.70% |
| AAV9 | 0.20% | 7.42% |

### EXAMPLE 4

### Targeting of Transgenes to AAVS1 in Mouse ES Cells

The foregoing observation that ES cells could be infected with AAV2 prompted the initiation of infection-based integration essays. The transgenes to be integrated, GFP and the neomycin resistance gene, were provided by recombinant single strand AAV2, whereas Rep, responsible for targeting the transgenes, was provided in trans by means of co-infection with wtAAV2. In brief, CCE mouse ES cells cultured on gelatin were co-infected with wt AAV and recombinant AAV2 at an MOI of 10⁶. Cells were passaged onto fresh neomycin-resistant feeders 48 hours after infection, and G418 selection (300 mg/ml) was started. Five days after the start of selection, G418-resistant colonies were aspirated and expanded. Finally, cells were harvested for FACS analysis and genomic DNA extraction. In this experiment, infections were performed in 96-well plates, and 6 clones were generated of which 3 were GFP-positive. Transgene integration analysis was focused on clone 4, as FACS analysis of this clone showed a single population of GFP-expressing cells.

Direct PCR and an unbiased linker-mediated PCR technique (Schroder et al. (2002) Cell 110: 521-529; Wu et al. (2003) Science 300: 1749-1751) were used to detect where the transgene had integrated. Both strategies showed that the transgene in clone 4 was targeted to AAVS1, 8429 bp downstream of the TRS/RBS motifs. PCR results indicated that wt AAV sequences are absent in the genome of this targeted clone.

Southern blot analysis showed disruption of Mbs85, the gene that is embedded in AAVS1. A different blot indicated that rAAV2 only integrated in AAVS1, since hybridization with a GFP probe resulted in a single band that cohybridized with the disrupted Mb85 band. Control DNA hybridized against a genomic MBS85 probe revealed the about 6.5kb undisrupted AAVS1 fragment. After removal of the MBS85 probe and hybridization to an rAAV-specific probe, the Southern blot indicated a single rAAV integration event with a vector genome fragment that co-migrates with the disrupted MBS85 fragment

AAVS1-targeted mouse ES cells show normal in vitro differentiation capacities and continue to express GFP throughout differentiation. Clone 4 ES cells were grown on gelatin for two passages in order to deplete feeders, trypsinized and cultured in non-adherent conditions to allow for the formation of EBs. It was found that EB differentiation occurred normally while GFP expression remains unchanged. At day 4, EBs expressed Flk-1 and c-kit profiles indicative of normal differentiation.

The Following Differentiation Assays were Performed on Targeted Mouse ES Cells.

1. Blast Colony-Forming Assay

This assay supports the growth of the hemangioblast, a precursor with the potential to generate both hematopoietic and endothelial lineages. These bipotential precursors represent a transient population that develops between day 3.0 and day 3.25 of differentiation and persists for 12-18 hours. These times can vary by 3-6 hours, depending on the batch of FCS and on the ES cell line used. The embroyoid body (EB)-derived hemangioblasts grow in response to VEGF and generate colonies consisting of cells with undifferentiated blast-cell morphology (Keller G. M., Webb S., and Kennedy M. in Methods in Molecular Medicine, vol. 63: Hematopoietic Stein Cell Protocols)

Targeted ES cells were differentiated in standard serum-containing conditions, EBs were harvested and dissociated at day 3.5 and added to a blast-methylcellulose (MEC: 1%, D4T (embryonic endothelial cell line) conditioned medium 25%, FCS 10%, Glutamine 1%, transferrin 300 μg/ml, ascorbic acid 25 μg/ml, monothioglycerol 4×10⁻⁴M, VEGF 5 ng/ml, I1-6 10 ng/ml, IMDM up to 100%) assay.

Blast colonies detected 3 days after initiation of the assay had typical morphology and maintained uniform GFP expression.

The assay is described by Kouskoff et al. (2005) Proc. Natl. Acad. Sci. 102:13170-5.

2. Cardiomyocyte Assay

Targeted ES cells were differentiated in standard serum-containing conditions, EBs were harvested and dissociated at day 4 and re-aggregated for 20 hours in serum-free conditions (StemPro34, L-Glutamine 2 mM, transferrin 200 μg/ml, ascorbic acid 0.5 mM, monothioglycerol 4.5×10⁻⁴M, VEGF 5 ng/ml, bFGF (30 ng/ml). Aggregates were transferred to gelatin-coated dishes containing StemPro34, L-Glutamine 2 mM, VEGF 5 ng/ml, bFGF (30 ng/ml). Three days later, beating cardiac clusters were observed. These clusters maintained uniform GFP expression.

3. Neuronal Differentiation Assay

Targeted ES cells were first depleted of feeders in N2B27 medium. After the second round of feeder depletion, cells were harvested and transferred to gelatin-coated dishes containing N2B27 medium and 0.3% MTG. Medium was changed daily. Neuron-like cell types were visible after 12 days of culture. Neuronal morphology was confirmed by immunohistochemistry using the neuron-specific marker Tuj1 (anti-tubulin bIII). Uniform GFP expression was observed in tubulin bIII-expressing neurons. The assay is adapted from Ying et al. (2003) Methods Enzymol. 365:32741.

Integration assays performed on CCE and Hela cells. The first clone analyzed for site-specific integration carried the transgenes in AAVS1. In order to address the issue of frequency, pools of CCE cells that were either infected with single-stranded wt and recombinant AAV2, with single-stranded rAAV2 alone or with single-stranded wtAAV2 alone were generated. For the cells that were infected with rAAV2, both in the absence and presence of wtAAV2, the population was split up in cells that were selected with G418 and cells that were not selected with G418. Respectively, 11 and 6 clones were aspirated from the pools of rAAV2− and wt+rAAV2-infected cells. One of those clones, ‘clone 4 (r+wt)’ was analyzed. Since it was important to compare the obtained integration frequencies with those obtained from a human cell line that previously had been shown to support AAV-mediated site-specific integration, an integration assay was performed in Hela cells using the same conditions as used for CCE cells. The number of GFP-positive cells in the pools that were infected with rAAV2 alone or with wt and rAAV2 are in the same range for both mouse ES cells and human Hela cells (see table 3).

TABLE 3

| | CCE | Hela |
|---|---|---|
| rAAV2 - no selection | 0.15% | 0.21% |
| r+wtAAV2 - no selection | 0.08% | 0.35% |
| rAAV2 - G418 selection | 26.09% | 2.11% |
| r+wtAAV2 - G418selection | 81.72% | 77.41% |

This table shows the number of GFP-positive cells, determined by FACS analysis respectively 4 and 5 passages after infection, in the absence and presence of selection.

As can be seen in Table 3, the number of GFP-positive cells increased dramatically when cells are coinfected with wtAAV2. Non-selected CCE cells are an exception.

The foregoing example demonstrates that a) AAV-mediated targeted gene delivery can be achieved into the mouse AAVS1 ortholog, b) targeted gene delivery to this locus is feasible in ES cells, c) as determined to date, disruption of AAVS1 does not interfere with multilineage in vitro differentiation of ES cells and d) that transgene expression is maintained throughout differentiation.

### EXAMPLE 5

### Infection of Human ES Cells with AAV

Maintenance and infection of hES cells. Human ES cells (WAO1) were maintained on irradiated mouse embryonic feeder cells in DMEM-F12 medium (L-Glutamine 1 mM, non-essential amino acids 1%) containing 20% serum replacement (Knockout-Invitrogen), 4 ng/ml basic Fibroblast Growth Factor, and beta mercaptoethanol (0.1 mM). Cultures were monitored daily and cells were passaged every 4-5 days. For passaging, ES cells were trypsinized (0.25% trypsin, 0.1% EDTA) for 3 minutes; the trypsin removed and replaced with medium containing 50% FBS and 50% F12 medium and Matrigel™ (0.2%). Then, cells were resuspended and washed. Approximately 25% of the cells were replated on fresh feeder cells. Cells were maintained in 37° C. incubators at 5% CO₂. Using this protocol healthy bES colonies that are alkaline phosphatase and c-kit positive were obtained. Minimal cell death occurred during the passaging process.

In addition, in order to determine transduction efficiencies that were not influenced by the presence of mouse feeder cells, growth conditions on Matrigel™ were established. Using this approach the colonies were maintained for several passages without significant differentiation.

For feeder depletion prior to infection, ES cells were cultured for 1 passage in wells of 6-well plates coated with Matrigel™ (Becton Dickinson—growth factor-reduced, diluted 1:1 in DMEM).

Cells were harvested from this culture vessel, counted and seeded in Matrigel™-coated, serum-free medium-containing 96-well plates at a density of approximately 10,000 cells per well.

24-48 hours later, cells from a couple of representative wells were counted in order to calculate the amount of virus needed to infect every well at a multiplicity of infection of 10⁶. ES cells were then infected with single or double strand recombinant AAV-GFP viruses, resuspended in 30 □1 of serum-free F12 medium. Infections were performed at 37° C. in the presence or absence of adenovirus; plates were shaken by hand every 15 minutes. Adenovirus was included in these experiments in order to first assess virus uptake without the contribution of downstream roadblocks as for example second-strand synthesis that has previously been shown to influence transduction rate. After 1 hour, 70 □1 of fresh medium was added and plates were placed back in the incubator. ES cells were incubated for 72 hours while daily replacing 75% of the medium with fresh medium.

72 hours post-infection cells were harvested for flow cytometry and the number of GFP-positive cells was determined.

Table 4 shows the results of these experiments.

TABLE 4
Infection of WA01 cells by AAV serotypes
Table 4. Small hES colonies that were seeded on mouse feeder cells
were infected by AAV serotypes 1, 2 and 5 and, where indicated,
superinfected by adenovirus (MOI: 500) at multiplicities of 10³ to
10⁵ genomes per cell. The data are given as percent GFP
positive cells that were gated on a population enriched for
hES cells. FACS analysis was performed 72 h post infection.

| | 1000 | 1000 | 10,000 | 10,000 | 100,000 | 100,000 |
|---|---|---|---|---|---|---|
| | −Ad | +Ad | −Ad | +Ad | −Ad | +Ad |
| rAAV1 | 0 | 0 | 0 | 0 | 0.19 | 0.31 |
| rAAV2 | 0 | 0.38 | 0.18 | 0.65 | 0.79 | 3.27 |
| rAAV5 | 0 | 0 | 0 | 0 | 0 | 0 |

Based on these initial data that highlighted the preference of AAV2 for infection of WA01 cells under these conditions, further optimization of the procedures utilized this AAV serotype.

Optimization of infection conditions. In further infection studies, adenovirus was excluded from the infection mixture. Multiplicity of infection was increased to 10⁶ genomes per cell. When infection was performed in the presence of mouse feeder cells, c-kit labeling was included in the FACS analysis in order to exclude contributions by the mouse cells. In addition, the following conditions were tested: WA01 cells were infected either on a mouse feeder layer, on Matrigel™ or in suspension (Table 5). Subsequent to infection the suspension cells were plated on mouse feeder cells. Further analysis of the cells that had been infected in suspension showed a significant change in morphology, as also confirmed by forward/sideward scatter in FACS analysis. In this analysis it also became apparent that c-kit had been down-regulated as a result of this particular condition. These changes could not be observed in cells that were infected on feeders or on Matrigel™. Based on these observations and the results shown in Table 5 further infection experiments were performed on cells that were maintained on Matrigel.

TABLE 5
Transduction of WA01

| | Single strand AAV2 | Double strand AAV2 |
|---|---|---|
| H1 on | 5.58 ± 0.51 | n.d. |
| feeders | (n = 4) | |
| H1 in | 14.89 ± 2.40 | n.d. |
| suspension | (n = 4) | |
| H1 on | 17.98 ± 1.86 | 42.82 ± 4.04 |
| Matrigel | (n = 4) | (n = 4) |

WA01 (H1) cells were infected by dsAAV and ssAAV2 based viruses using three different conditions for cell growth and maintenance.

An additional variable shown in Table 5 was the inclusion of double-strand (or self-complementary) AAV2 that in a number of previous studies had shown enhanced transduction efficiencies. As can be seen, on average nearly 43% of WA01 cells can be transduced on Matrigel™ when dsAAV2 is used. The FACS analysis of WA01 cells infected with dsAAV2 was performed. Also c-kit expression levels of mock-infected and dsAAV2-infected cells were compared. The c-kit expression levels were comparable in both conditions. In addition, cells infected with dsAAV2 were passaged onto fresh feeder cells and continued to show normal growth characteristics.

### EXAMPLE 6

### Infection of Human ES Cells with AAV

Human embryonic stem cells (HES2 cells) were maintained on mouse embryonic feeders using the same protocol as described for WA01 cells hereinabove.

HES2 cells were transduced with recombinant AAV1, 2, 5, 8 and 9, respectively. The viruses were “pseudotyped”, i.e. these vectors contain the AAV2 ITRs and the identical transgene as used hereinabove. These genomes were packaged into the AAV1, 2, 5, 8 and 9 capsids, respectively. In these experiments both single-stranded (ss) as well as double-stranded (ds) vectors were used. Infections at an MOI of 10⁶ were performed on small human ES colonies (HES2), cultured on Matrigel™. Transduction efficiency was determined as the number of GFP expressing cells present in the cultures 48 hours post-infection. As can be seen in Table 6, with the exception of AAV5 (ss and ds), infections of HES2 cells with ds vectors of all serotypes resulted in significant transduction.

TABLE 6
Transduction efficiencies on HES2 cells: percentage of
GFP-expressing cells as determined by flow cytometry

| | Single-stranded | Double-stranded |
|---|---|---|
| AAV1 | 0.16% | 14.15% |
| AAV2 | 8.31% | 45.91% |
| AAV5 | 0.00% | 0.03% |
| AAV8 | 0.45% | 11.7% |
| AAV9 | 0.06% | 2.21% |

### EXAMPLE 7

### Targeting of Transgenes to AAVS1 in Human ES Cells

WAO1 cells were grown on Matrigel™ and co-infected with single-stranded wt AAV and recombinant AAV2, containing the hGFP gene and a neomycin resistance cassette, flanked by the AAV terminal repeats (MOI 10⁶). Cells were passaged onto fresh feeders 48 hours after infection, and G418 selection was started. It had previously been determined that G418 selection at 50 μg/ml left the feeder cells undisturbed, but killed off mock-infected hES. Mouse embryonic fibroblasts (feeder cells) grown in serum-free medium do not tolerate higher concentrations of G418, which they do when grown in serum-containing medium. Two weeks after the start of selection, healthy-looking G418-resistant colonies had developed. Aspiration techniques that were used to isolate mouse ES clones also worked for hES as in an independent experiment 15 wt AAV-infected hES clones were generated.

Forty G418-resistant clones were aspirated and expanded on Matrigel™ in order to isolate genomic DNA. This DNA was then digested with EcoRI or HindIII and run on a Southern blot. Hybridization with a hAAVS1-specific probe showed disruption of the target site in 2 out of the 28 thus far analyzed WAO1 clones, indicating that the AAVS1 locus has been targeted.

### EXAMPLE 8

### Generation of Chimeric Animals

Three wells of a six-well plate were coated with gelatin and irradiated mouse embryonic fibroblasts. 2-3 days before the blastocyst injections, one frozen vial of amplified targeted ES cells was thawed and plated into the earlier prepared three wells. On the day of injection, the medium was changed to medium without LIF, 1-2 hours before the cells were used. The cells were then trypsinized, pelleted and resuspended in 10 ml of DMEM supplemented with 20 mM HEPES (pH 7.3) and 10% FCS.

Blastocysts were obtained from immature (4 week old) B6D2F1 female mice which had been superovulated with PMS and HCG, followed by matings with C57B1/6 males. Three days after plugs were identified in these females, the mice were sacrificed by CO₂ overdose. The uterus was isolated from each animal, and blastocysts were flushed from each uterus. Isolated blastocysts were then injected with targeted ES cells. These injected blastocysts were reimplanted into the uterus of pseudopregnant females and mated two days before the day of blastocyst microinjection. Generally, 12-15 microinjected blastocysts were reimplanted into each host female. The reimplantation surgeries were done under avertin anesthesia, and topical 1% lidocaine was administered immediately after the surgery. After the animals recovered from the surgery, they were returned to the animal room where most of them delivered their pups 17 days after the reimplantation of embryos. Approximately 1 week after delivery of pups derived from microinjected blastocysts, the coat color becomes apparent on the pups, and it is this coat color which is used to determine the relative success of the experiment. The ES cells used for these studies were derived from a mouse strain (129) which has agouti coat color, while the donor blastocysts were obtained from black mice (C57B1/6). The experiment is judged successful if coat color chimeras are observed in which the agouti color (dominant to black) makes up to at least 50% of the animal's coat color. In this example, 6 chimeric animals (2 males and 4 females) were born; based on coat color, the percentage of chimerism was estimated at 30-50%.

In order to assess transgene expression from AAVS1 in vivo, blood was collected from female mice and labeled with a pan-leukocyte marker (CD 45.2). CD 45.2 positive cells were analyzed for GFP expression using FACS analysis. 4 -8.5% of the leukocytes expressed GFP, demonstrating that transgenes were expressed from AAVS1 throughout differentiation in vivo. Furthermore, no apparent deleterious effects of integration into AAVS1 and the resulting genomic disruption were observed.

1. A method for site-specific integration of a transgene into the genome of an embryonic stem (ES) cell comprising introducing into the ES cell an adeno-associated virus (AAV) vector comprising a transgene, and a Rep protein or a nucleic acid encoding a Rep protein.

2. The method of claim 1 wherein the ES cell is a human ES cell.

3. The method of claim 1 wherein the ES cell is a mouse ES cell.

4. The method of claim 1 wherein the AAV vector comprises a pair of AAV inverted terminal repeats flanking a transgene under the control of a promoter.

5. The method of claim 1 wherein the AAV inverted terminal repeats are AAV2 inverted terminal repeats.

6. The method of claim 1 wherein the AAV vector comprises an AAV capsid.

7. The method of claim 6 wherein the AAV capsid comprises capsid proteins selected from the group consisting of AAV1, AAV2, AAV3, AAV4, AAV5, AAV6, AAV7, AAV8 and AAV9 proteins and combinations thereof.

8. The method of claim 6 wherein the AAV capsid comprises AAV2 capsid proteins.

9. The method of claim 1 wherein the Rep protein is Rep 68 or Rep 78.

10. The method of claim 1 wherein the Rep protein comprises the amino-terminal 208 amino acids of Rep 78.

11. The method of claim 1 wherein the nucleic acid encoding a Rep protein is introduced into the ES cell in trans to the AAV vector.

12. The method of claim 1 wherein the nucleic acid encoding a Rep protein is introduced into the ES cell in cis to the AAV vector.

13. The method of claim 4 wherein the AAV vector comprises a nucleic acid encoding a Rep protein sited outside the inverted terminal repeats.

14. The method of claim 1 wherein the AAV vector is single-stranded.

15. The method of claim 1 wherein the AAV vector is double-stranded.

16. The method of claim 1 wherein one of the inverted terminal repeats has a deletion of the terminal resolution site.

17. A method for site-specific integration of a transgene into the genome of a stem cell comprising introducing into said stem cell an AAV vector comprising a transgene, and a Rep protein or a nucleic acid encoding a Rep protein.

18. The method of claim 16 wherein said stem cell is an adult stem cell.

19. The method of claim 17 wherein said stem cell is selected from the group consisting of a hematopoietic stem cell, bone marrow stromal stem cell, adipose derived adult stem cell, olfactory adult stem cell, neuronal stem cell and skin stem cell.

20. (canceled)

21. An embryonic stem cell having a transgene integrated at the AAVS1 locus.

22. A differentiated cell generated from an embryonic stem cell having a transgene integrated at the AAVS1 locus.

23. The differentiated cell of claim 22 selected from the group consisting of a hematopoietic cell, endothelial cell, cardiomyocyte, skeletal muscle cell and neuronal cell.

24. (canceled)

25. A transgenic nonhuman animal comprising a transgene integrated into AAVS1.

26. The transgenic animal of claim 25 wherein the animal is a mouse.

27-30. (canceled)


SEQUENCE LISTING (2 sequences)

SEQ ID NO: 1 - DNA, 53 bp, Homo sapiens
ggttggggctcggcgctcgctcgctcgctgggcgggcgggcggtgcgatgtcc

SEQ ID NO: 2 - DNA, 59 bp, Mus musculus
tgttcgtcgcccggtgctcgctcgctcgcttagtaggcgggcgagcggccggcgcgatg